(19) United States
(12) Patent Application Publication
(10) Pub. No.: US 2018/0070840 A1
(43) Pub. Date: Mar. 15, 2018

(54) WEARABLE DEVICES WITH TWO STAGE OF USE

(71) Applicant: KONINKLIJKE PHILIPS N.V., EINDHOVEN (NL)

(72) Inventors: JOHN CRONIN, BONITA SPRINGS, FL (US); JOSEPH BODKIN, WILLISTON, VT (US); GERARD DE HAAN, HELMOND (NL); WILLEM VERKRUIJSSE, VELDHOVEN (NL)

(21) Appl. No.: 15/559,070

(22) PCT Filed: Mar. 16, 2016
(86) PCT No.: PCT/EP2016/055629

§ 371 Date: Sep. 18, 2017

Related U.S. Application Data
(60) Provisional application No. 62/135,400, filed on Mar. 19, 2015.

(30) Foreign Application Priority Data
Aug. 13, 2015 (EP) 15181002.5

(51) Int. Cl. A61B 5/024; A44C 5/14; A61B 5/00; G06F 1/26

(52) U.S. Cl. CPC A61B 5/02438; A44C 5/14; A61B 5/4806; A61B 5/1118; G06F 1/266; A61B 5/681; A61B 5/7435

(57) ABSTRACT

A two-state wearable device may include a wearable band (e.g., a watch band) and a display module (e.g., a watch face). In a “detached” state, the wearable band and display module act as separate devices, and the display module can be charging its battery while the wearable band gathers sensor data (e.g., sleep tracking data) and provides a vibrating/audio alarm clock function. In an “attached” state, the wearable band and display module act together as a single device, meaning that the data collected by the wearable band during the “detached” state is sent to and stored at the display module, and that sleep settings input at a user interface of the display module impact the wearable band's alarm function. The display module may also use its newly-recharged battery to recharge the wearable band's battery so that it can be used the next night.

### TECHNICAL FIELD

The present invention generally relates to wearable technology. More specifically, the present invention relates to wearable devices that include a functional wearable band that may releasably attach a display module.

### BACKGROUND

Wearable electronic devices, or as used herein, wearable technology is a new class of electronic systems that can provide data acquisition through a variety of unobtrusive sensors that may be worn by a user. The sensors gather information, for example, about the environment, the user's activity, or the user's health status. However, there are significant challenges related to the coordination, computation, communication, privacy, security, and presentation of the collected data. Additionally, there are challenges related to power management given the current state of battery technology. Furthermore, analysis of the data is needed to make the data gathered by the sensors useful and relevant to end-users. In some cases, additional sources of information may be used to supplement the data gathered by the sensors. The many challenges that wearable technology presents require new designs in hardware and software.

Wearable technology may include any type of mobile electronic device that can be worn on the body, attached to or embedded in clothes and accessories of an individual and currently existing in the consumer marketplace. Processors and sensors associated with the wearable technology can display, process or gather information. Such wearable technology has been used in a variety of areas, including monitoring health of the user as well as collecting other types of data and statistics. These types of devices may be readily available to the public and may be easily purchased by consumers. Examples of some wearable technology in the health arena include the FitBit, the Nike Fuel Band, the Jawbone Up, and the Apple Watch.

Typically, a wearable device can be used to gather data about the user. For example, a wearable device can use one or more sensors to monitor health parameters (e.g., heart rate) of a user during the day, and data relating to sleep patterns while the user is asleep at night. Thus, many wearable devices provide useful functions at any point in time during the day. However, wearable devices are typically small devices, having small batteries that must be charged often, sometimes requiring a charge after less than 24 hours of use. As a result, users of such wearable devices must typically decide between sacrificing nighttime use of the wearable device (e.g., to generate sleep pattern data) in order to charge the device, or using the wearable device at night (e.g., to generate sleep pattern data) but charging the wearable device during the day or potentially running out of battery charge during the day.

### SUMMARY

Accordingly, various embodiments disclosed herein are directed to a two-state wearable device including a wearable band, e.g., a watch band, and a display module, e.g., a watch face. In a “detached” state, the wearable band and display module act as separate devices, and the display module can be charging its battery while the wearable band gathers sensor data (e.g., sleep tracking data) and/or provides a vibrating/audio alarm clock function. In an “attached” state, the wearable band and display module act together as a single device, such that the data collected by the wearable band during the “detached” state is sent to and stored at the display module, and that sleep settings input at a user interface of the display module impact the wearable band's alarm function. In an embodiment, the display module may also use its newly-recharged battery to recharge the wearable band's battery so that it can be used the next night.

According to an embodiment, a wearable device with two states of use, the wearable device comprises: a wearable band configured to releasably attach a display module such that the wearable band is configurable between an attached state and a detached state, wherein, in the attached state, the wearable band is attached to and in communication with the display module, and in the detached state, the wearable band is separate and apart from the display module, the wearable band comprising: a wearable band processor configured to execute program instructions and configured to determine when the wearable band is in the detached state or the attached state; a wearable band battery configured to power the wearable band, at least when in the detached state, and to receive a charge from an external power source; at least one wearable band sensor configured to perform at least one sensor measurement at least when in the detached state; a wearable band memory configured to store at least a portion of the wearable band sensor measurements at least when in the detached state; and a wearable band communication interface configured for transmitting at least a portion of the wearable band sensor measurements to the display module at least when in the attached state.

According to an embodiment, the display module, being configured to releasably attach to the wearable band, comprises: a display module processor configured for executing program instructions and configured to determine whether the display module is attached to the wearable band; a display module communication interface configured for receiving at least a portion of the wearable band sensor measurements from the wearable band; and a display module memory configured to receive and to store the wearable band sensor measurements; and a display module battery configured to power the display module.

According to an embodiment, the external power source is the display module battery.

According to an embodiment, the external power source is an external connectable battery.

According to an embodiment, the display module is a watch face.

According to an embodiment, the display module is configured to set at least one setting of wearable band.

According to an embodiment, the display may further comprise one or more display module sensors configured for performing one or more display module sensor measurements.

According to an embodiment, the wearable band processor is further configured to execute wearable band instructions to generate an alarm output at a predetermined time, the alarm output being one of a vibration of a vibrator of the wearable band or an audio output from a speaker of the wearable band.

According to an embodiment, the wearable band processor is further configured to execute wearable band instructions to: process received sleep settings input, the sleep settings input including at least the predetermined alarm time, and transmit the sleep settings input to the wearable band after determining that the display module is connected to the wearable band.

According to an embodiment, the wearable band sensor measurements include sleep measurements related to at least one of sleep quality, sleep duration, sleep movements, sleep patterns, sleep interruptions, sleep pulse, sleep blood pressure, sleep breathing, a time value related to a user falling asleep, or a time value related to a user waking up.

According to an embodiment a method for utilizing a wearable device with two stages of use, comprises: providing a wearable band configured to releasably attach a display module such that the wearable band is configurable between an attached state and a detached state, wherein, in the attached state, and in the detached state, the wearable band is separate and apart from the display module, wherein the wearable band comprises a wearable band processor, a wearable band battery configured to power the wearable band, a wearable band memory, and at least one wearable band sensor; performing at least one wearable band sensor measurements by the at least one wearable band sensors included in the wearable band; storing at least a portion of wearable band sensor measurements in the wearable band memory; determining whether the wearable band is reconnected to the display module; and transmitting the wearable band sensor measurements from the wearable band memory to the display module.

According to an embodiment, the method further comprises the step of charging a battery of the display module of the wearable device when the display module has been detached from a wearable band of the wearable device, by a power source.

According to an embodiment, the method further comprises the step of charging the wearable band battery by the display module.

According to an embodiment, the method further comprises the step of charging the wearable band battery by an external source.

According to an embodiment, the display module is a computerized watch face.

According to an embodiment, the wearable band sensor measurements are related to at least one of sleep quality, sleep duration, sleep movements, sleep patterns, sleep interruptions, sleep pulse, sleep blood pressure, sleep breathing, a time value related to a user falling asleep, or a time value related to a user waking up.

According to an embodiment, the method further comprises the step of setting, using the display module, at least one setting of wearable band.

According to an embodiment, the step of performing at least one display module sensor measurement by at least one display module sensor.

According to an embodiment, a wearable device with two states of use comprises: a wearable band configured to releasably attach a display module such that the wearable band is configurable between an attached state and a detached state, wherein, in the attached state, the wearable band is attached to and in communication with the display module, and in the detached state, the wearable band is separate and apart from the display module, the wearable band comprising: a wearable band processor configured to execute program instructions and configured to determine when the wearable band is in the detached state or the attached state; a wearable band battery configured to power the wearable band, at least when in the detached state, and to receive a charge from an external power source; at least one wearable band sensor configured to perform at least one sensor measurement at least when in the detached state; a wearable band memory configured to store at least a portion of the wearable band sensor measurements at least when in the detached state; a wearable band communication interface configured for transmitting at least a portion of the wearable band sensor measurements to the display module at least when in the attached state, wherein the display module, being configured to releasably attach to the wearable band, comprises: a display module processor configured for executing program instructions and configured to determine whether the display module is attached to the wearable band; a display module communication interface configured for receiving at least a portion of the wearable band sensor measurements from the wearable band; and a display module memory configured to receive and to store the wearable band sensor measurements.

According to an embodiment, the external power source is the display module battery.

In various implementations, a processor or controller may be associated with one or more storage media (generically referred to herein as “memory,” e.g., volatile and non-volatile computer memory such as RAM, PROM, EPROM, and EEPROM, floppy disks, compact disks, optical disks, magnetic tape, etc.). In some implementations, the storage media may be encoded with one or more programs that, when executed on one or more processors and/or controllers, perform at least some of the functions discussed herein. Various storage media may be fixed within a processor or controller or may be transportable, such that the one or more programs stored thereon can be loaded into a processor or controller so as to implement various aspects of the present invention discussed herein. The terms “program” or “computer program” are used herein in a generic sense to refer to any type of computer code (e.g., software or microcode) that can be employed to program one or more processors or controllers.

In one network implementation, one or more devices coupled to a network may serve as a controller for one or more other devices coupled to the network (e.g., in a master/slave relationship). In another implementation, a networked environment may include one or more dedicated controllers that are configured to control one or more of the devices coupled to the network. Generally, multiple devices coupled to the network each may have access to data that is present on the communications medium or media; however, a given device may be “addressable” in that it is configured to selectively exchange data with (i.e., receive data from and/or transmit data to) the network, based, for example, on one or more particular identifiers (e.g., “addresses”) assigned to it.

The term “network” as used herein refers to any interconnection of two or more devices (including controllers or processors) that facilitates the transport of information (e.g. for device control, data storage, data exchange, etc.) between any two or more devices and/or among multiple devices coupled to the network. As should be readily appreciated, various implementations of networks suitable for interconnecting multiple devices may include any of a variety of network topologies and employ any of a variety of communication protocols. Additionally, in various networks according to the present disclosure, any one connection between two devices may represent a dedicated connection between the two systems, or alternatively a non-dedicated connection. In addition to carrying information intended for the two devices, such a non-dedicated connection may carry information not necessarily intended for either of the two devices (e.g., an open network connection). Furthermore, it should be readily appreciated that various networks of devices as discussed herein may employ one or more wireless, wire/cable, and/or fiber optic links to facilitate information transport throughout the network.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE DRAWINGS

FIG. 1A illustrates a two-state wearable device in an attached state, according to an embodiment.

FIG. 1B illustrates a two-state wearable device in a detached state, according to an embodiment.

FIG. 2 illustrates a two-state wearable device architecture including a wearable band and a display module, according to an embodiment.

FIG. 3 is a flow diagram illustrating an operation of a sleep software as executed by a wearable band, according to an embodiment.

FIG. 4 illustrates a wearable band database stored in the memory of a wearable band, according to an embodiment.

FIG. 5 is a flow diagram illustrating an operation of a display module base software as executed by a display module, according to an embodiment.

FIG. 6 illustrates a computing device architecture that may be utilized to implement the various features and processes described herein, according to an embodiment.

FIG. 7 illustrates a display module database stored in the memory of a display module, according to an embodiment.

FIG. 8 illustrates a sleep graphical user interface (GUI) as executed by a display module, according to an embodiment.

FIG. 9 is a flow diagram illustrating an operation of a sleep software as executed by a display module, according to an embodiment.

FIG. 10 illustrates a flow chart of a method, according to an embodiment.

### DETAILED DESCRIPTION

Several of the embodiments referenced in appended drawings are now explained. The following description and drawings are illustrative and are not to be construed as limiting. Numerous specific details are described to provide a thorough understanding of various embodiments. However, in certain instances, well-known or conventional details are not described in order to provide a concise discussion of embodiments.

Reference in the specification to “one embodiment” or “an embodiment” means that a particular feature, structure, or characteristic described in conjunction with the embodiment can be included in at least one embodiment. The appearances of the phrase “in one embodiment” or “in an embodiment” in various places in the specification do not necessarily all refer to the same embodiment.

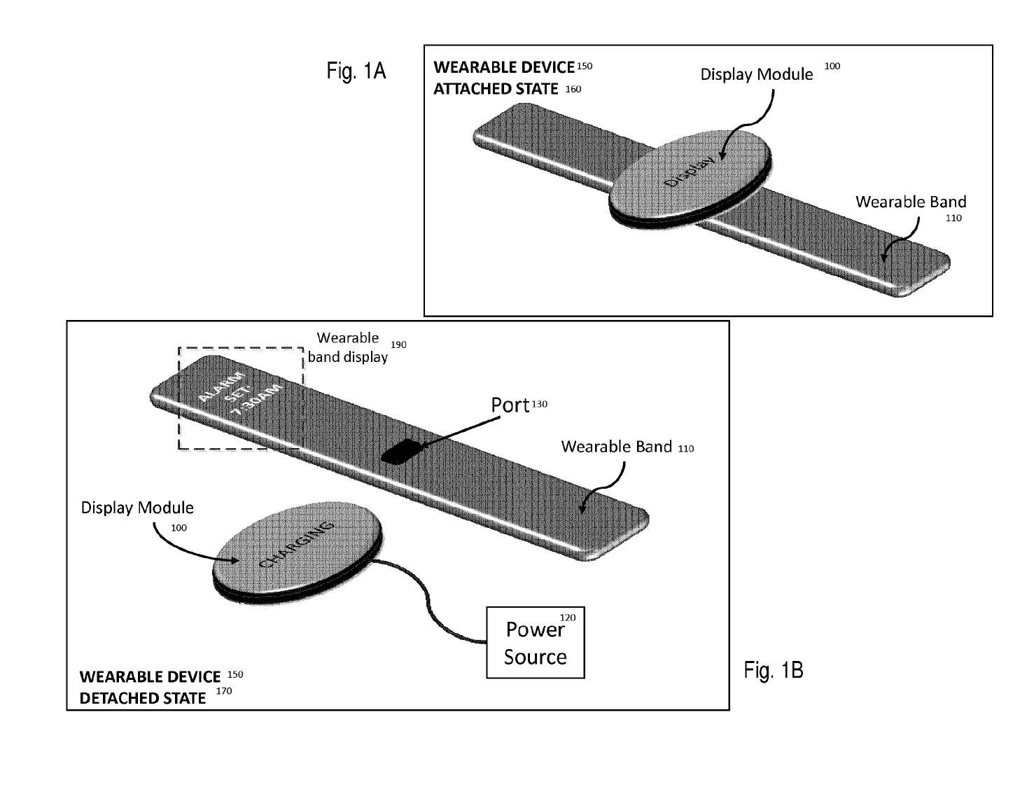

Referring now to the figures, FIG. 1A and FIG. 1B illustrate two states of an embodiment of a two-state wearable device 150, characterized by having an attached state 160 and a detached state 170. FIG. 1A illustrates an embodiment of two-state wearable device 150 in an attached state 160. In the attached state 160, a display module 100 (e.g., a computerized watch face) is attached to a wearable band 110 (e.g., a computerized watch band). For the purposes of this application only, the terms “state” and “stage” are interchangeable and refer to position of display module 100 as attached to wearable band 110 are detached from wearable band 110.

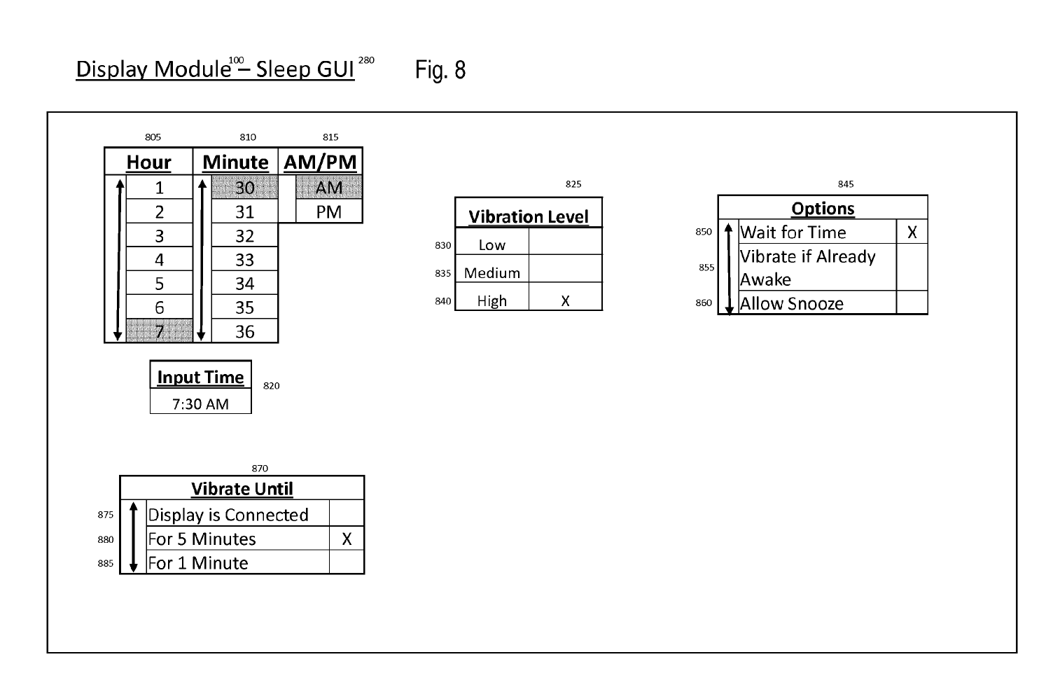

In an embodiment, attached state 160 may be associated with a usage period in which it is useful to have more active sensors. For example, attached state 160 may be associated with a daytime usage period of the wearable device 150. For example, during the daytime, display module 100 may provide the user, through the display 245 of display module 100, messages and information which may not be accessed or viewed when the user is asleep, such as time displays, weather displays, email displays, SMS/text message displays, instant message displays, phone call displays, video call displays, calendar event displays, reminder displays, purchase displays, mail tracking displays, fitness tracking displays, health tracking displays, health warning displays, sleep tracking displays (regarding a past night's sleep or a daytime nap), and graphical user interfaces (GUIs) for typing responses and adjusting settings (e.g. , sleep alarm settings as discussed further in relation to FIG. 8). Display module 100 may also include other user-responsive features, such as microphone voice input functionality. Display module 100 may also include various sensors, such as health sensors (e.g., measuring heart rate or blood pressure) or fitness sensors (e.g., measuring steps walked or ran) or environmental sensors (e.g., measuring air temperature or humidity).

Wearable band 110 of wearable device 150 may, in some cases, and in various embodiments, be designed to use less power than display module 100. This may be accomplished a number of ways. For example, wearable band 110 may forego a display entirely, or may use a low-powered wearable band display 190 (or at least a lower-powered display than display module 100), or may use a simplified processor 290 as compared to the more powerful processor 295 display module 100. Alternately, wearable band 110 need not be designed to use less power than display module 100, and may as a result include a full-featured display 190 (e.g. a touchscreen color display 190) and/or a processor 290 that is as powerful or more powerful than the processor 295 of display module 100. The wearable band 110 may also include sensors, such as health sensors (e.g., measuring heart rate or blood pressure) or fitness sensors (e.g., measuring steps walked or ran) or environmental sensors (e.g., measuring air temperature or humidity). In an embodiment, the outputs of the sensors of wearable band 110, and other data, may then be fed to display module 100 through the port 130 illustrated in FIG. 1B while wearable device 150 is in attached state 160 illustrated in FIG. 1A. In an alternate embodiment, wearable band 110 may communicate data to display module 100 through a wireless connection, such as a Bluetooth connection, a radio-frequency connection, an inductive connection, a Wi-Fi direct communication, or a near-field communication, while in attached state 160, or even while in detached state 170 if wearable band 110 is within wireless range of display module 100.

hile in attached state 160, in an embodiment, wearable band 110 of wearable device 150 may also receive an electric charge from display module 100. In other words, an electric charge from battery 255 of display module 100 may be used to recharge battery 230 of the wearable band. Alternatively, wearable band 110 may provide an electric charge to display module 100 (e.g., an electric charge from battery 230 of wearable band 110 may be used to recharge the battery 255 of display module 100). (In an alternate embodiment, wearable band 110 may receive a charge from an external source such as a charger which may be plugged into the wall or from an external connectable battery.)

FIG. 1B illustrates an embodiment of a two-state wearable device 150 in a detached state 170. In detached state 170, display module 100 is detached from wearable band 110. This may be useful, for example, to charge display module 100 while wearable band 110 continues to operate and obtain sensor measurements via sensors 220 of the wearable band.

In an embodiment, detached state 170 may, for example, be associated primarily with a time period in which it may be useful to charge wearable device 150, but during which it may still be useful to obtain sensor measurements. Coupling the entire wearable device 150 to a power source 120 (e.g., using a charger 200) generally involves cables or close proximity to a wireless charging station. A user who wishes to continue using the wearable device 150 in some capacity (e.g., sleep tracking) cannot comfortably or reliably continue to use a wearable device while it is connected to a power source 120 in order to charge. A sleeping user may unknowingly entangle themselves using a charging cable, potentially endangering the user's life. A sleeping user similarly cannot be relied upon to keep his/her hand in a particular position while sleeping in order to continue receiving an electric charge from a proximity-based wireless charging station. Discomfort caused by such attempts to charge a device while a user is asleep may cause the user's sleep quality and duration to worsen, defeating the purpose of using a sleep tracking device. Alternatively, wearable band 110 may be charged while display module 100 is attached to and worn with a different band (either a smart wearable band 110 as described herein, or a normal band). Alternatively, display module could be worn with a lanyard, belt clip, pouch, etc, while wearable band 110 is charging.

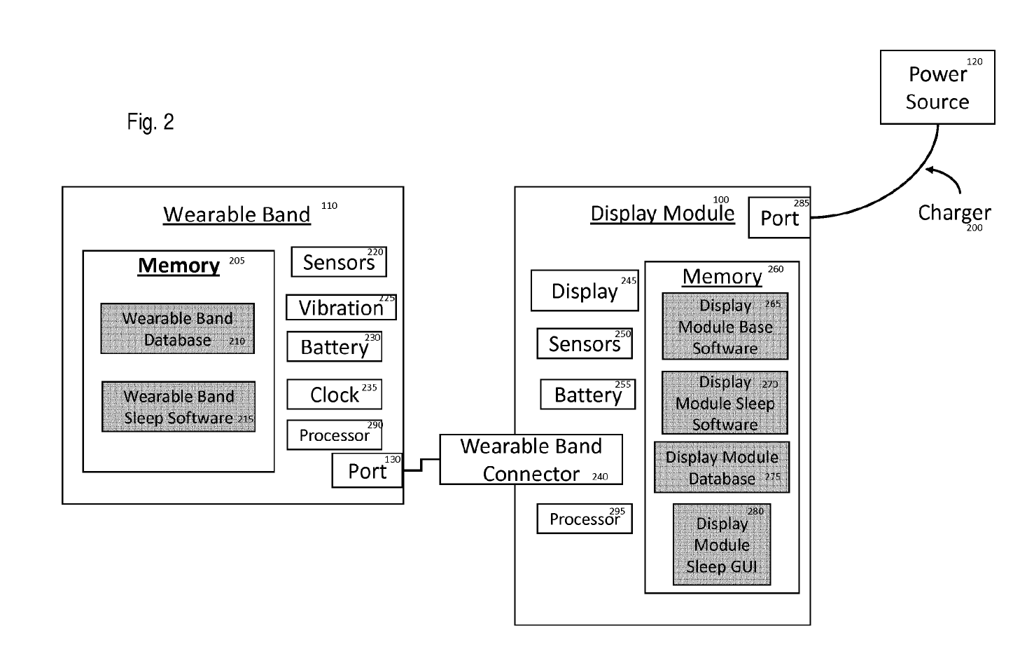

Therefore, detached state 170 may be used to charge display module 100 while allowing wearable band 110 to retain certain functions such as a sleep-tracking function. Sleep-tracking function may be provided by sensors 220 of wearable band 110 and memory 205 of wearable band 110 (and processor 290 of wearable band 110 where applicable) while display module 100 is detached and charging. During detached state 170, for example, wearable band 110 may obtain (e.g., according to wearable band sleep software 215 of FIG. 2) sensor measurements from sensors 220 of wearable band 110 and store the measurements into memory 205 (e.g., in wearable band database 210 of FIG. 2). Sensor measurements from sensors 220 can be related to sleep quality, sleep duration, sleep movements, sleep patterns, sleep interruptions, sleep pulse, sleep blood pressure, sleep breathing, a time value related to a user falling asleep, or a time value related to a user waking up. Sensor measurements from sensors 220 can also measure other quantities, such as blood pressure, pulse, breathing, or any other possible sensor measurements discussed in relation to FIG. 2, or as are known in the art and advantageous for addition to wearable device 150.

Wearable band 110 and display module 100 may then be reconnected into the attached state 160 of the wearable device 15, such as depicted in FIG. 1A, in the morning or once display module 100 has finished charging. Any sensor measurements (or a portion of sensor measurements) obtained by wearable band 110 sensors 220 and stored by wearable band 110 in memory 205 may then be transferred over to display module 100 for storage (e.g., at the display module database 275 of FIG. 2) and/or interpretation (e.g., by the display module sleep software 270 of FIG. 2). Display module 100 may then also use its newly recharged battery 255 (with electric charge from power source 120) to transmit electric charge to the battery 230 of wearable band 110 until wearable band 110 is ready to be used again (e.g., the following night). In an alternate embodiment, wearable band 110 may receive a charge from another external source (e.g. a charger) or from another external attachable battery. For example, wearable band 110 may receive, in addition to display module 100, a detachable battery which is configured to power and/or charge battery 230 (and/or battery 255), while wearable band 110 may continue to be worn by the user. Display module 100 may similarly receive a detachable battery which may be used to charge battery 255 of display module 100 or battery 230 of wearable band 110.

Wearable band 110 may also include a clock and a vibrator and/or a speaker. These can be used to wake up the user of wearable band 110 through a timed alarm or a “smart” alarm that wakes the user up only if the user is in “light” sleep (as opposed to “heavy” rapid-eye-movement “REM” sleep).

In an embodiment, while in either state, (in an alternate embodiment, in the attached state) display module 100 may be used to adjust settings. These settings may adjust, for example, which sensors 220 are to be used by wearable band 110, a period of time between sensor measurements of the sensors 220, setting of alarms to be executed by wearable band 110 at a particular time, setting of “smart” alarms to be executed by wearable band 110 that use the sensor measurements of sensors 220 take into account what stage of sleep the user is in (e.g., avoiding waking the user while the user is in REM sleep), adjusting the sound/noise of an alarm to be executed by wearable band 110, adjusting the vibration intensity of an alarm to be executed by wearable band 110, synchronizing clocks between display module 100 and wearable band 110, and other settings of the sleep tracking functionality of wearable band 110. Alternately, the settings may adjust the same values as related to the sensors 250 of display module 100 and alarms to be executed by display module 100. In an alternate embodiment, these settings may be adjusted by a third-party device, such as a computer, a mobile device, etc., which may be paired with display 100 or directly with wearable band 110.

The settings may be adjusted through a sleep graphical user interface (GUI) 280 at display module 100 regardless of the state of two-state wearable device 150 (e.g., attached 160 or detached 170). These settings may then be transmitted to wearable band 110 the next time two-state wearable device 150 is joined into attached state 160 (e.g., through the port or wirelessly). Alternately, the settings from the sleep GUI 280 may be transmitted wirelessly to wearable band 110 from display module 100 in detached state 170 (e.g., transmission triggered periodically or by a user input). Similarly, the two-state wearable device 150 may perform a clock synchronization between display module 100 and wearable band 110 either when the two-state wearable device 150 is joined into the attached state 160 (e.g., through the port or wirelessly) or wirelessly in the detached state 170 (e.g., synchronization triggered periodically or by a user input).

FIG. 2 illustrates an embodiment of a two-state wearable device architecture including a wearable band 110 and a display module 100. In an embodiment, the two-state wearable device architecture may include at least a wearable band 110, a display module 100, and a power source 120.

As have been partially described in conjunction with FIGS. 1A and 1B, wearable band 110 may include various components, such as, for example, one or more wearable band sensors 220, a vibrator 225 (“vibration”), a processor 290, a power storage unit 230 (“battery”) (e.g., a rechargeable battery or a replaceable battery), a clock, a memory 205, and a communication/power port/module 130 (“port”). Memory 205 of wearable band 110 may include a wearable band database 210 and a wearable band sleep software 215, or other software (such as fitness software) according to various other embodiments and uses of wearable band 110. Wearable band architecture illustrated in FIG. 2 should be interpreted as illustrative rather than limiting, and other embodiments may include additional or different components or elements stored in memory, or may lack illustrated components or elements stored in memory.

Communication/power port/module 130 of wearable band 110 may be a wired connection module (e.g., a USB port module, a FireWire port module, a Lightning port module, a Thunderbolt port module, customized audio jack port module, a magnetic charging cable port module, or a proprietary cable port module), a physical connection module (e.g., communicative and/or electrical-power-providing contact through a direct physical contact of a metallic lead from wearable band 110 to display module 100), or a wireless communicative and/or electrical-power-providing connection module. The wireless communicative and/or electrical-power-providing connection module capabilities may be split or combined, and may include a wireless communication module (e.g., a Wi-Fi connection module, a 3G/4G/LTE cellular connection module, a Bluetooth connection module, a Bluetooth low energy connection module, a Bluetooth Smart connection module, a near field communication module, a radio wave communications module) as well as a wireless electrical power module (e.g., a magnetic induction charging module or a magnetic resonance charging module).

In an embodiment, the one or more wearable band sensors 220 of wearable band 110 may include sensors 220 for measuring blood pressure, heart rate, body temperature (e.g., thermometer), blood sugar or glucose, acceleration e.g., accelerometer), insulin, vitamin levels, respiratory rate, heart sound (e.g., microphone), breathing sound (e.g., microphone), movement speed, steps walked or ran (e.g., pedometer), skin moisture, sweat detection, sweat composition, nerve firings (e.g., electromagnetic sensor), or similar health measurements. In some embodiments, additional sensors 220 may also measure allergens, air quality, air humidity, air temperature, and similar environmental measurements.

Display module 100 may include a display 245, one or more display module sensors 250, a power storage unit 255 (“battery”) (e.g., a rechargeable battery or a replaceable battery), a memory 260, a processor 295, a communication/power port/module 240, and a charging port/module 285.

In some embodiments, wearable band communication/power port/module 130 may be the same type of communication/power port/module as the display module communication/power port/module 240, or may be of a compatible type such that the wearable band port/module 130 can be connected to display module port/module 240 in a manner that allows electrical communications and/or electrical power charge to be transferred between wearable band 110 and display module 100 in either one or both directions. In an alternate embodiment, wearable band 110 may communicate power to and/or from display module 100 via an additional port (not shown). Memory 260 of display module may include a display module base software 265, a display module sleep software 270, a display module database 275, and a display module sleep graphical user interface (GUI) 280. Communication/power port/module 240 of the display module 110 may include any of the types of communication/power ports/modules described in relation to the communication/power port/module 130 wearable band 110. The communication/power port/module 240 need not be the same type of communication/power port/module as the communication/power port/module 130. Display module 100 architecture illustrated in FIG. 2 should be interpreted as illustrative rather than limiting, and other embodiments may include additional or different components or elements stored in memory, or may lack illustrated components or elements stored in memory 260.

The charging port/module 285, in an embodiment, may be a wired power-receiving connection module (e.g, a USB port module, a FireWire port module, a Lightning port module, a Thunderbolt port module, a customized audio jack port module, a magnetic power cable port module, or a proprietary power-cable connector module), a physical charging module (e.g., charging through a direct physical contact of a metallic lead from wearable band 110 the power source), or a wireless charging module (e.g., a magnetic induction charging module or a magnetic resonance charging module).

The one or more sensors of display module 100 may include, in an embodiment, sensors for measuring blood pressure, heart rate, body temperature (e.g., thermometer), blood sugar or glucose, acceleration e.g., accelerometer), insulin, vitamin levels, respiratory rate, heart sound (e.g., microphone), breathing sound (e.g., microphone), movement speed, steps walked or ran (e.g., pedometer), skin moisture, sweat detection, sweat composition, nerve firings (e.g., electromagnetic sensor), or similar health measurements. In some embodiments, additional sensors may also measure allergens, air quality, air humidity, air temperature, and similar environmental measurements.

One of ordinary skill will appreciate, in conjunction with a review of this disclosure, that display module 100 may have the same, or different, sensors as wearable band 110. In an embodiment, the sensors of wearable band 110 may be of a type suitable for the purpose of wearable device 150 in detached state 160 (e.g., sleep monitoring or fitness tracking) while the sensors of the display may be suitable for the purposes of wearable device in the attached state. Of course, these are just examples, and any sensor may be used which are advantageous for purposes intended for each state. One of ordinary skill will further appreciate that sensors of wearable band 110 may be sized to fit into the band (i.e. small or flexible sensors). Furthermore, when in the attached state, if any sensors are duplicative between the wearable band 110 and display module 100 (i.e. both have a motion sensor), one of each duplicate may be turned off to conserve power. Alternatively, when in the attached state, all or a portion of sensors in wearable band 110 may be turned off to conserve power. In yet another embodiment, when in the attached state, different sensors in display module 100 and wearable band 110 may be used to augment and improve the sensing power of each or to work together to improve health monitoring, etc. Finally, when in the attached state (or connected via a wireless data connection), display module 100 may be configured to directly control and read the outputs of sensors located in wearable band 110. Conversely, wearable band 110 may be able to directly control and read the outputs of sensors located in display module 100 when connected or in the attached state.

Power source 120 may be any type of power source, such as a standard wall socket (e.g., supplying power a predetermined voltage), a generator, a battery (e.g., a portable battery device or a car battery). The charger 200 may include a cable (e.g, a USB cable, a FireWire cable, a Lightning cable, a Thunderbolt cable, customized audio jack cable, a magnetic charging cable, or a proprietary cable), an adapter adapting the wall socket current to a particular voltage and/or amperage and/or type of current (e.g., alternating current or direct current), and/or a wireless charging dock/cradle/mat/area/volume. Similarly, display module 100 may be configured to receive an additional, releasably attachable battery in order to charge battery 255. The releasable battery may then be detached to be, itself, charged. In an alternate embodiment, battery 255 may be removed from display module 100 for charging. While battery 255 is detached from display module 100, a second battery may keep module 100 powered. In this way, charged batteries may be swapped in and out of display module 100.

In some embodiments, wearable band 110 may include a wearable band display 190, which may be a low-energy display (e.g., light emitting diode display). Though this is not shown in FIG. 2, an embodiment of a low-energy display 190 is illustrated in an embodiment of wearable band 110 of FIG. 1B (displaying “ALARM SET: 7:30 AM”).

An embodiment of the operation of two-state wearable device 150 may be, for example, a user using two-state wearable device 150 during the course of the day, and taking various different sensor readings from sensors 250 of display module 100 and/or the sensors 220 of wearable band 110 of wearable device 150 in its attached state 160. At the end of the day, when the user decides to charge the two-state wearable device 150, the user may input the time they wish to wake up on sleep GUI 280 (which is then transferred to wearable band 110), but instead of removing the whole two-state wearable device 150, the user only removes display module 100, leaving on wearable band 110. This changes the “state” of wearable device 150 from the attached state 160 to the detached state 170. Display module 100 then charges its power storage unit 255 (e.g., rechargeable battery) using the power source 120 throughout the night while wearable band 110 (with its own power storage unit 230, sensors 220, clock 235, alarm, and vibrator 225) stays powered on and operating, providing the user with sensor readings (e.g., measuring the user's sleep behavior) using wearable band sensors 220. In the morning, wearable band 110 vibrates to wake the user according to the sleep settings from the sleep GUI 280, and the user reattaches the now-charged display module 100 to wearable band 110 to take the wearable device 150 from the detached state 170 into attached state 160. Wearable band 110 then transmits the sensor data that it recorded from sensors 220 during the night to display module 100, to be added to the display module database 275. Display module 100 then (or simultaneously, or beforehand) provides electrical energy/charge transferred from its battery 255 to recharge wearable band's battery 230 so that wearable band 110 may function again once the wearable device 150 is returned to the detached state 170 the following night.

In an embodiment, two-state wearable device 150 may also be adapted for fitness purposes. For example, wearable band 110 may be used by the user while display module 100 stays in a locker to prevent it from getting damaged by impacts or water damage. Toward this end, wearable band 110 may be made waterproof even if display module 100 is not. In some embodiments, however, display module 100 may also be waterproof.

Two-state wearable device 150 may also include multiple wearable bands 110 per display module 100. For example, different wearable bands 110 may have different purposes (e.g., a separate sleep wearable band 110 and a separate fitness wearable band 110) and different integrated sensors to accomplish those purposes. In some embodiments, wearable bands 110 could be given out or sold at an event, for example, and include special event software related to that event (e.g., a wearable band with integrated LED lights of a particular color for a music-related event, a cultural rally such as an independence day celebration, or a political event). Wearable bands 110 may also be offered in a variety of styles and colors to suit the fashion, comfort, or fitness needs of a particular user, or to match with a particular event. Further, wearable bands 110 and/or display module 100 may be offered in a variety of materials, including plastic, silicone, or metal. In some embodiments, a user could connect wearable band 110 and/or display module 100 to a third party device (e.g., the user's computer, the user's mobile device, a doctor's computer, a doctor's mobile device, or a doctor's wearable device 150) in order to transfer data to the third party device (e.g., the wearable band database 210 and/or display module database 275 and/or the sleep GUI sleep settings), receive data from the third party device, or synchronize data with the third party device. Having multiple wearable bands 110 may also allow a user to swap out one band for a fully charged wearable band, if the charge is beginning to get low.

In some embodiments, display module 100 may be a complex computer device with diverse capabilities. In other embodiments, display module 100 may feature a simple user interface (e.g., only displaying time) and may function more as a battery pack for wearable band 110 than as a separate device in its own right. In such embodiments, the base software 265, sleep software 270, database 275, and sleep GUI could alternately be stored in the wearable band memory 205 and/or executed by the wearable band processor 290 instead of being stored in the display module memory 260 and executed by the processor 295.

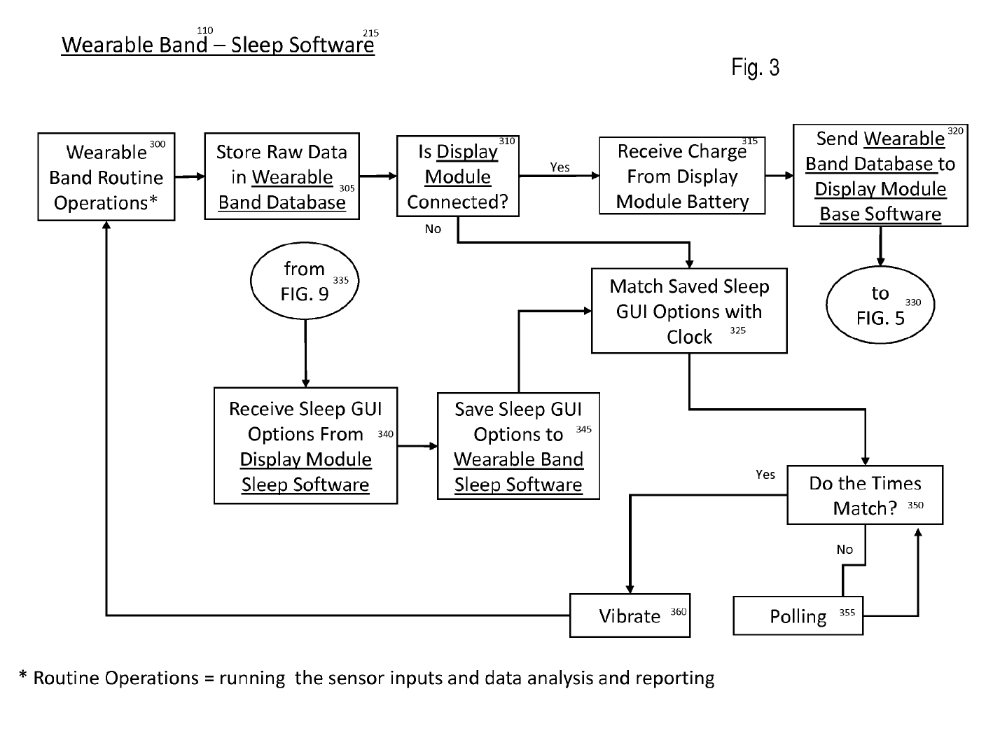

FIG. 3 is a flow diagram illustrating an embodiment of the operation of a sleep software as executed by an embodiment of wearable band 110. The sleep software controls various functions relating to the sleep tracking functionality of wearable band 110 and the connection between wearable band 110 and display module 100.

In an embodiment, the sleep software's operations begin with wearable band 110 performing routine operations in step 300. In the case of wearable band 110, routine operations may mean routine sleep tracking operations, namely receiving sensor measurement inputs from the one or more sensors 220 (e.g., tracking motion, heart rate, breathing, or any of the other possible sensor inputs described in relation to an embodiment of wearable band 110 of FIG. 2) of wearable band 110. The routine operations may also include data analysis and reporting (e.g., calculating sleep quality or sleep stage based on the sensor inputs). Wearable band 110 then stores the raw data from the sensor measurement input and/or from the data analysis/reporting into the memory 205 of wearable band 110 (e.g., in the wearable band database 210) in step 305.

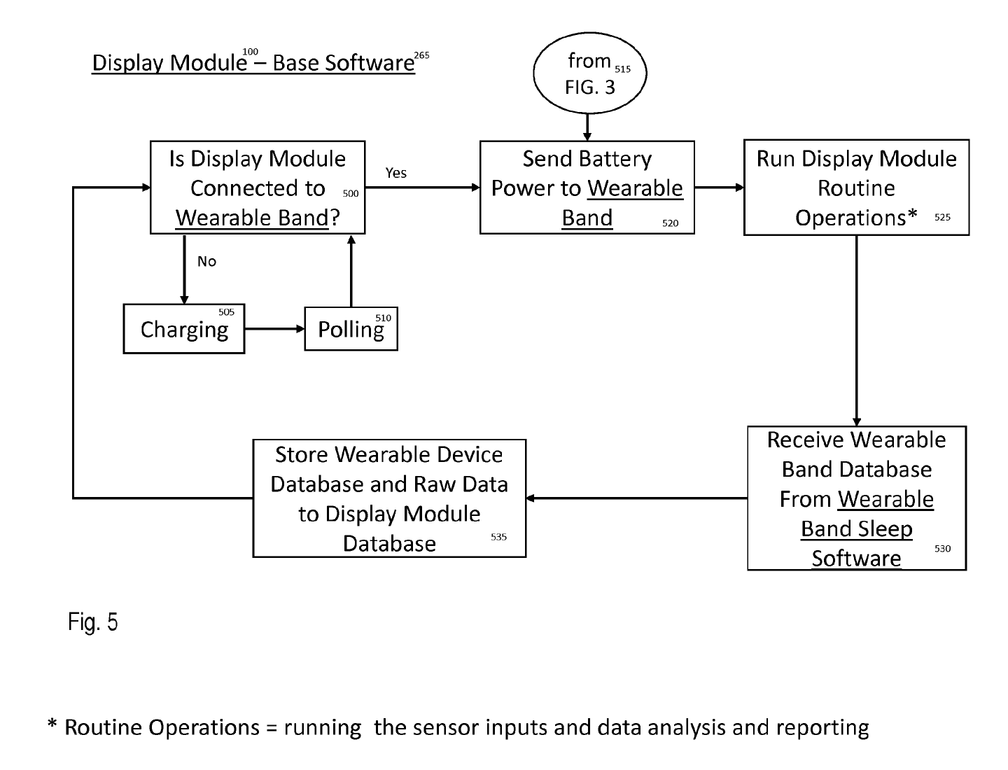

In an embodiment, wearable band 110 then polls to determine if display module 100 is connected to wearable band 110 in step 310 (e.g., to see if the two-state wearable device 150 is in the attached state 160 illustrated in FIG. 1A as opposed to the detached state 170 of FIG. 1B). If wearable band 110 is connected to display module 100, wearable band 110 then may receive an electric charge to the wearable band's power storage unit 230 (e.g. rechargeable battery) from display module's power storage unit 255 (e.g., a rechargeable battery or replaceable battery) in step 315. Alternately, wearable band 110 may transmit an electric charge from the wearable band's power storage unit 255 (e.g. a rechargeable battery or replaceable battery) to the display module's power storage unit 230 (e.g., a rechargeable battery). In step 320, wearable band 110 may then transmit at least a subset of the wearable band database 210 (or the sensor measurement and/or data analysis/reporting data in some other format) to display module 100, which may then receive and process the wearable band database 210 using the display module's base software 265 in step 330 (described in further detail with respect to FIG. 5). In an alternate embodiment, instead of checking whether display module 100 is attached, display module 110 may notify wearable band 110 of the attachment by pushing an attachment notification to wearable band 110. For example, display module may access and change an internal variable of wearable band 110, notifying wearable band 110 of the connection.

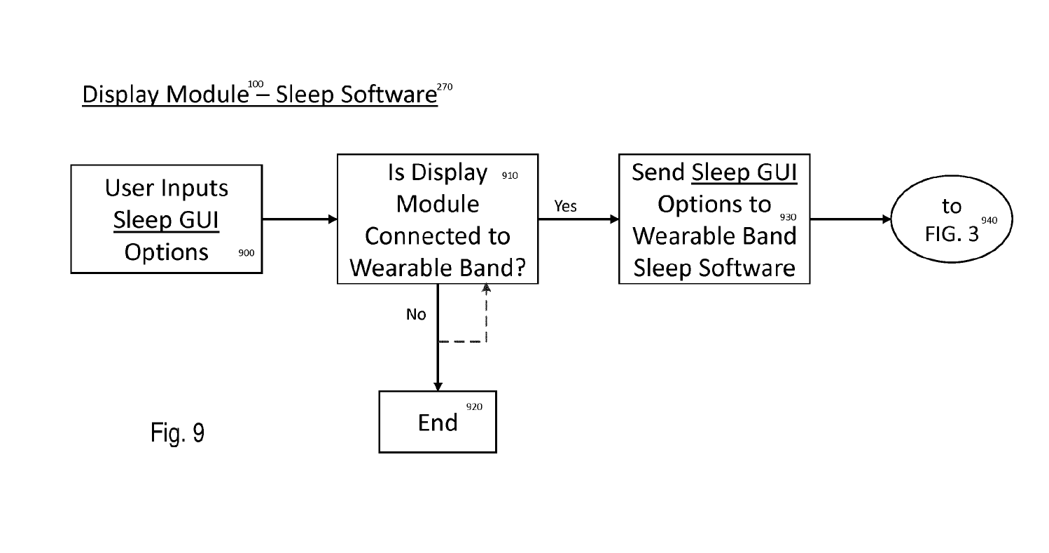

If, when wearable band 110 checked whether display module 100 was connected in step 310, display module 100 was not found to be connected (e.g., the two-state wearable device 150 is in the detached state 170 illustrated in FIG. 1B as opposed to the attached state 160 of FIG. 1A), wearable band 110 may check the sleep settings that it has previously obtained from the display module's sleep settings GUI 280 (or from a third party device), and match these sleep settings against the clock of wearable band 110 to check if any of these settings indicate that an action should be undertaken by wearable band 110 in step 325. These sleep settings may have been input using the display module's sleep settings GUI 280 in step 335 (described in further detail with respect to FIG. 9), transmitted from the display module's sleep software 270 and received at the wearable device 150 in step 340, and saved to the wearable band sleep software 215 of wearable band 110 in step 345. If, when wearable band 110 checks the times and sleep settings to determine whether an action should be undertaken by wearable band 110 in step 350, there is no matching sleep setting wearable band 110 may continue to periodically poll to check for a matching sleep setting indicating an action should be undertaken by wearable band 110 in step 355. In some embodiments, wearable band 110 may also return to wearable band routine operations in step 300, or to checking to see if display module 100 is connected in step 310.

If wearable band 110 does locate a matching sleep setting indicating an action should be undertaken by wearable band 110 in step 350, wearable band 110 may then alert the user in step 360. This alert may take the form of a vibration from the vibrator of wearable band 110 in step 360, or an audio clip played from the speaker(s) of wearable band 110, or a text notification displayed on the display 190 of wearable band 110, or a graphical notification displayed on the display 190 of wearable band 110, or a video notification played using the display 190 and/or speaker(s) of wearable band 110, or some combination thereof The matching sleep setting may be, for example, an alarm or a “smart” alarm. While the flow diagram in FIG. 3 shows a particular order of operations performed by certain embodiments, it should be understood that such order is (e.g., alternative embodiments can perform the operations in a different order, combine certain operations, overlap certain operations, etc.).

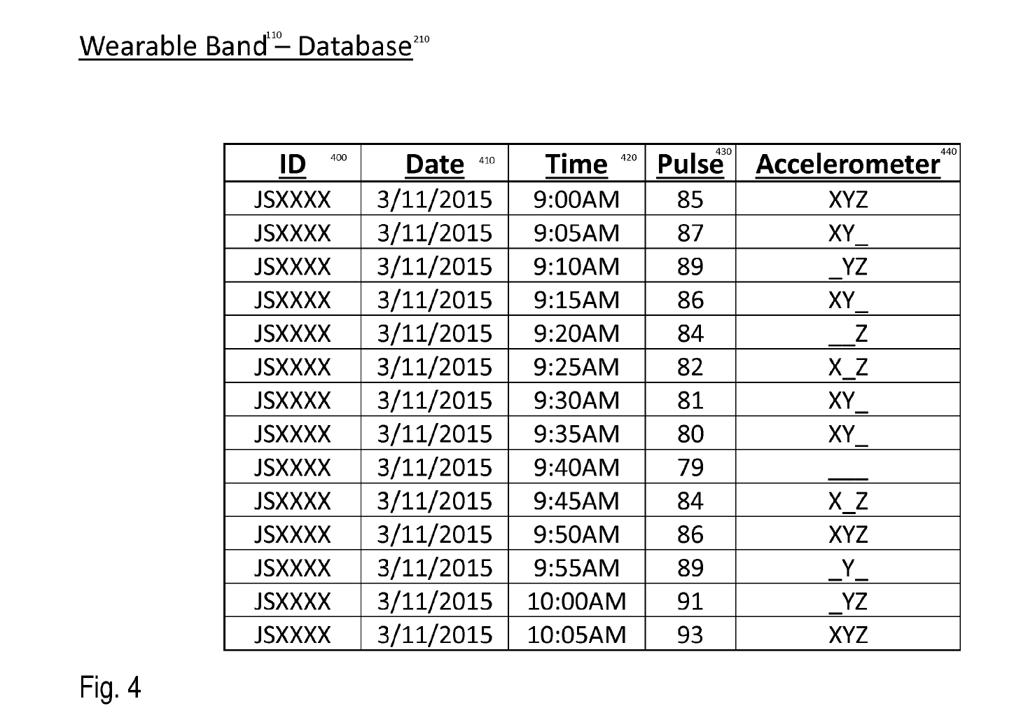

FIG. 4 illustrates an embodiment of wearable band database 210 stored in the memory 205 of an embodiment of wearable band 110. An embodiment of wearable band database 210 may include multiple columns relating to sensor measurements from wearable band sensors 220 (columns, as described herein, are only examples of categories of data stored in database 210). Some columns may correspond to a user identification (ID) 400, a measurement date 410, a measurement time 420, and a variety of sensor measurements corresponding to the marked date and time. Sensor measurements may include, for example, measurements from a pulse sensor 430 and measurements from an accelerometer 440.

In this embodiment of wearable band database 210, all of the entries pertain to a user with the user ID “JSXXXX” (e.g., “ID” column 400). In other instances, not all entries must belong to the same user—for instance, a wearable device 150 may include multiple User IDs corresponding to multiple users who may use the wearable device 150 (e.g., different members of a family may switch off using the same wearable device 150, and data corresponding to different users may be marked differently in “ID” column 400). For example, all of the measurements shown in FIG. 4 were taken on Mar. 11, 2015 (3/11/2015) (e.g., “Date” column 410). The database 210 may in some instances store data from multiple days. In some cases, the database 210 may be cleared out when data from the database 210 is transferred to display module 100. As shown, the measurements in the database 210 were taken every five minutes on March 11 between the hours of 9:00 AM and 10:05 AM (e.g., “Time” column 420). This may be adjusted using a settings interface such as the sleep GUI 280 or another interface. The pulse measurements ranged between a low of 79 (at 9:40 AM) and a high of 93 (at 10:05 AM) (e.g., “Pulse” column 430). The accelerometer tracks movement across the measured timespan, indicating when movement is detected in the X direction and/or Y direction and/or Z direction with “X” and “Y” and “Z” indicators (e.g., “Accelerometer” column 440). In some instances, an accelerometer may measure more detailed movement data, such as distances or degrees of movement.

FIG. 5 is a flow diagram illustrating an embodiment of the operation of a display module base software 265 as executed by an embodiment of display module 100. Base software 265 may control various operations of two-state wearable device 150, of display module 100 in particular, and of the connection between display module 100 and wearable band 110.

First, base software 265 may check to see whether display module 100 is connected to wearable band 110 in step 500. If it is not connected to wearable band 110, display module 100 then charges its battery 255 if it is connected to the power source 120 in step 505, and periodically polls to see whether wearable band 110 has been connected to display module 100 in step 510. If display module 100 has been connected to wearable band 110 (e.g., FIG. 3 for more details regarding wearable band 110's role in this connection in step 515), display module 100 may send electric power from the battery 255 of display module 100 to the battery 230 of wearable band 110 (step 520). Alternatively, the display module's battery 255 may in some embodiments receive electric power from the battery 230 of wearable band 110, or from an external connected battery in alternate embodiments.

Display module 100 may then run routine operations in step 525. For example, these routine operations may include receiving sensor measurement inputs from the one or more sensors 250 (e.g., tracking motion, heart rate, breathing, or any of the other possible sensor inputs described in relation to a display module 100 of FIG. 2) of display module 100. The routine operations may also include data analysis and reporting (e.g., calculating calories burned based on the sensor inputs). Display module 100 may then receive wearable band database 210 from wearable band sleep software 215 in step 530. Display module 100 may then store wearable band database 210 and/or the raw sensor measurement data and/or raw data analysis/reporting data from the wearable band database 210 (e.g., sensor measurements from wearable band sensors 220) and/or from the display module's own sensors 250 into the display module's memory 260 (e.g., at the display module database 275) in step 535. Display module 100 may then return to checking if display module 100 is still connected to wearable band 110 in step 500.

While the flow diagram in FIG. 5 shows a particular order of operations performed by certain embodiments, it should be understood that such order is (e.g., alternative embodiments can perform the operations in a different order, combine certain operations, overlap certain operations, etc.).

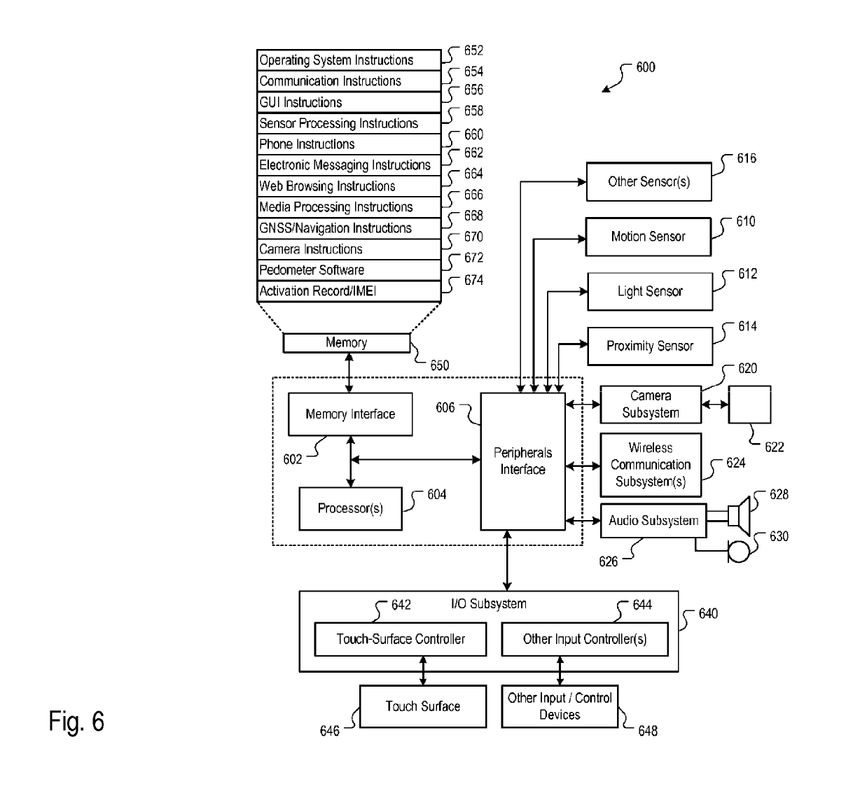

FIG. 6 illustrates an embodiment of a computing device architecture that may be utilized to implement the various features and processes described herein. For example, the computing device architecture 600 could be implemented in wearable band 110 and/or display module 100. Architecture 600 as illustrated in FIG. 6 includes memory interface 602, processors 604, and peripheral interface 606. Memory interface 602, processors 604 and peripherals interface 606 can be separate components or can be integrated as a part of one or more integrated circuits. The various components can be coupled by one or more communication buses or signal lines.

Processors 604 as illustrated in FIG. 6 is meant to be inclusive of data processors, image processors, central processing unit, or any variety of multi-core processing devices. Any variety of sensors, external devices, and external subsystems can be coupled to peripherals interface 606 to facilitate any number of functionalities within the architecture 600 of the exemplar mobile device. For example, motion sensor 610, light sensor 612, and proximity sensor 614 can be coupled to peripherals interface 606 to facilitate orientation, lighting, and proximity functions of the mobile device. For example, light sensor 612 could be utilized to facilitate adjusting the brightness of touch surface 646. Motion sensor 610, which could be exemplified in the context of an accelerometer or gyroscope, could be utilized to detect movement and orientation of the mobile device. Display objects or media could then be presented according to a detected orientation (e.g., portrait or landscape).

Other sensors could be coupled to peripherals interface 606, such as a temperature sensor, a biometric sensor, or other sensing device to facilitate corresponding functionalities. Location processor 615 (e.g., a global positioning transceiver) can be coupled to peripherals interface 606 to allow for generation of geo-location data thereby facilitating geo-positioning. An electronic magnetometer 616 such as an integrated circuit chip could in turn be connected to peripherals interface 606 to provide data related to the direction of true magnetic North whereby the mobile device could enjoy compass or directional functionality. Camera subsystem 620 and an optical sensor 622 such as a charged coupled device (CCD) or a complementary metal-oxide semiconductor (CMOS) optical sensor can facilitate camera functions such as recording photographs and video clips.

In an embodiment, communication functionality can be facilitated through one or more communication subsystems 624, which may include one or more wireless communication subsystems. Wireless communication subsystems 624 can include 802.x or Bluetooth transceivers as well as optical transceivers such as infrared. Wired communication system can include a port device such as a Universal Serial Bus (USB) port or some other wired port connection that can be used to establish a wired coupling to other computing devices such as network access devices, personal computers, printers, displays, or other processing devices capable of receiving or transmitting data. The specific design and implementation of communication subsystem 624 may depend on the communication network or medium over which the device is intended to operate. For example, a device may include wireless communication subsystem designed to operate over a global system for mobile communications (GSM) network, a GPRS network, an enhanced data GSM environment (EDGE) network, 802.x communication networks, code division multiple access (CDMA) networks, or Bluetooth networks. Communication subsystem 624 may include hosting protocols such that the device may be configured as a base station for other wireless devices. Communication subsystems can also allow the device to synchronize with a host device using one or more protocols such as TCP/IP, HTTP, or UDP.

Audio subsystem 626 can be coupled to a speaker 628 and one or more microphones 630 to facilitate voice-enabled functions. These functions might include voice recognition, voice replication, or digital recording. Audio subsystem 626 in conjunction may also encompass traditional telephony functions.

I/O subsystem 640 may include touch controller 642 and/or other input controller(s) 644. Touch controller 642 can be coupled to a touch surface 646. Touch surface 646 and touch controller 642 may detect contact and movement or break thereof using any of a number of touch sensitivity technologies, including but not limited to capacitive, resistive, infrared, or surface acoustic wave technologies. Other proximity sensor arrays or elements for determining one or more points of contact with touch surface 646 may likewise be utilized. In one implementation, touch surface 646 can display virtual or soft buttons and a virtual keyboard, which can be used as an input/output device by the user.

Other input controllers 644, in an embodiment, may be coupled to other input/control devices 648 such as one or more buttons, rocker switches, thumb-wheels, infrared ports, USB ports, and/or a pointer device such as a stylus. The one or more buttons (not shown) can include an up/down button for volume control of speaker 628 and/or microphone 630. In some implementations, device 600 can include the functionality of an audio and/or video playback or recording device and may include a pin connector for tethering to other devices.

Memory interface 602 can be coupled to memory 650. Memory 650 can include high-speed random access memory or non-volatile memory such as magnetic disk storage devices, optical storage devices, or flash memory. Memory 650 can store operating system 652, such as Darwin, RTXC, LINUX, UNIX, OS X, ANDROID, WINDOWS, or an embedded operating system such as VxWorks. Operating system 652 may include instructions for handling basic system services and for performing hardware dependent tasks. In some implementations, operating system 652 can include a kernel.

Memory 650 may also store communication instructions 654 to facilitate communicating with other mobile computing devices or servers. Communication instructions 654 can also be used to select an operational mode or communication medium for use by the device based on a geographic location, which could be obtained by the GPS/Navigation instructions 668. Memory 650 may include graphical user interface instructions 656 to facilitate graphic user interface processing such as the generation of an interface; sensor processing instructions 658 to facilitate sensor-related processing and functions; phone instructions 660 to facilitate phone-related processes and functions; electronic messaging instructions 662 to facilitate electronic-messaging related processes and functions; web browsing instructions 664 to facilitate web browsing-related processes and functions; media processing instructions 666 to facilitate media processing-related processes and functions; GPS/Navigation instructions 668 to facilitate GPS and navigation-related processes, camera instructions 670 to facilitate camera-related processes and functions; and instructions 672 for any other application that may be operating on or in conjunction with the mobile computing device. Memory 650 may also store other software instructions for facilitating other processes, features and applications, such as applications related to navigation, social networking, location-based services or map displays.

Each of the above identified instructions and applications can correspond to a set of instructions for performing one or more functions described above. These instructions need not be implemented as separate software programs, procedures, or modules. Memory 650 can include additional or fewer instructions. Furthermore, various functions of the mobile device may be implemented in hardware and/or in software, including in one or more signal processing and/or application specific integrated circuits.

Certain features may be implemented in a computer system that includes a back-end component, such as a data server, that includes a middleware component, such as an application server or an Internet server, or that includes a front-end component, such as a client computer having a graphical user interface or an Internet browser, or any combination of the foregoing. The components of the system can be connected by any form or medium of digital data communication such as a communication network. Some examples of communication networks include LAN, WAN and the computers and networks forming the Internet. The computer system can include clients and servers. A client and server are generally remote from each other and typically interact through a network. The relationship of client and server arises by virtue of computer programs running on the respective computers and having a client-server relationship to each other.

One or more features or steps of the disclosed embodiments may be implemented using an API that can define on or more parameters that are passed between a calling application and other software code such as an operating system, library routine, function that provides a service, that provides data, or that performs an operation or a computation. The API can be implemented as one or more calls in program code that send or receive one or more parameters through a parameter list or other structure based on a call convention defined in an API specification document. A parameter can be a constant, a key, a data structure, an object, an object class, a variable, a data type, a pointer, an array, a list, or another call. API calls and parameters can be implemented in any programming language. The programming language can define the vocabulary and calling convention that a programmer will employ to access functions supporting the API. In some implementations, an API call can report to an application the capabilities of a device running the application, such as input capability, output capability, processing capability, power capability, and communications capability.

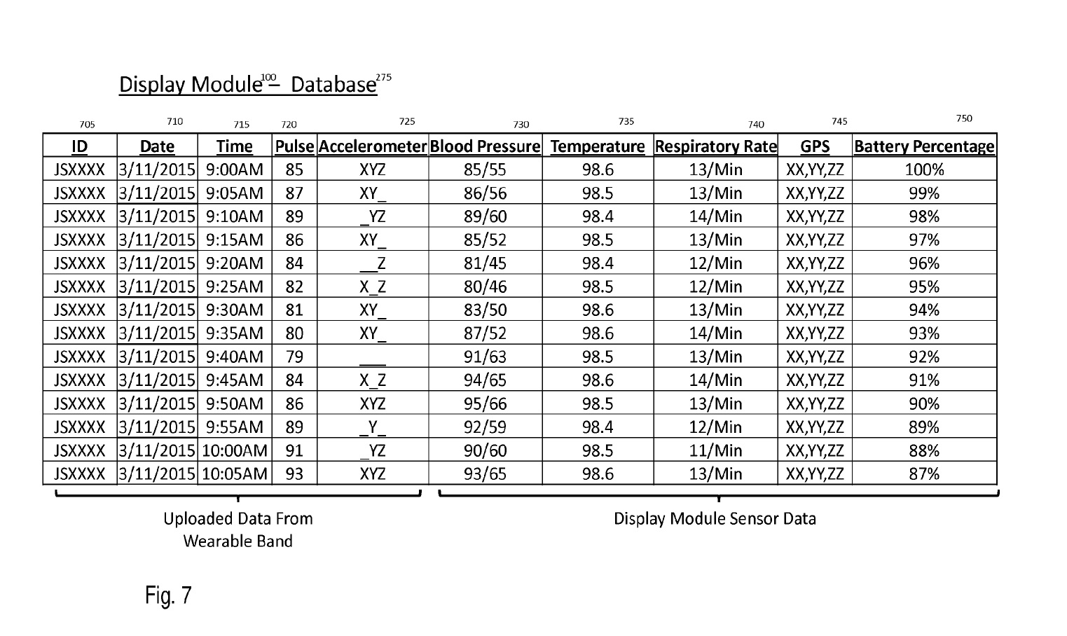

FIG. 7 illustrates an embodiment of a display module database 275 stored in the memory 260 of an embodiment of display module 100. This stores combined data from the sensors 220 of wearable band 110 and the sensors 250 of display module 100. In an embodiment, display module database 275 may include multiple columns, each column corresponding to a user identification (ID) 705, a measurement date 710, a measurement time 715 and a variety of sensor measurements corresponding to the marked date and time. The sensor measurements may include, for example, measurements from a pulse sensor 720, an accelerometer sensor 725, a blood pressure sensor 730, a body temperature sensor 735, and a respiratory rate sensor 740. The display module database 275 may include other data regarding one or both of wearable band 110 and display module 100, namely readings from a global positioning system or “GPS” location module 740 at the time/date of measurement, and readings from a battery percentage detector module 750 detecting a percentage of battery power remaining in the battery 255 and/or the battery 230.

The first five columns of the embodiment of display module database 275 of FIG. 7 are the same as those from the embodiment of wearable band database 210 of FIG. 4.

In an embodiment, all of the entries of display module database 275 of FIG. 7 pertain to a user with the user ID “JSXXXX” (e.g., “ID” column 705). In other instances, not all entries must belong to the same user—for instance, a wearable device 150 may include multiple User IDs corresponding to multiple users who may use the wearable device 150 (e.g., different members of a family may switch off using the same wearable device 150, and data corresponding to different users may be marked differently in “ID” column 705).

All of the measurements were taken on Mar. 11, 2015 (3/11/2015) (e.g., “Date” column 710). The database 275 may in some instances store data from multiple days. In some cases, the database 275 may be cleared out when data from the database 275 is transferred to display module 100.

The measurements in the database 275 were taken every five minutes on March 11 between the hours of 9:00 AM and 10:05 AM (e.g., “Time” column 715). This may be adjusted using a settings interface such as the sleep GUI 280 or another interface.

The pulse measurements ranged between a low of 79 (at 9:40 AM) and a high of 93 (at 10:05 AM) (e.g., “Pulse” column 720). In an embodiment, the accelerometer tracks movement across the measured timespan, indicating when movement is detected in the X direction and/or Y direction and/or Z direction with “X” and “Y” and “Z” indicators (e.g., “Accelerometer” column 725). In some instances, an accelerometer may measure more detailed movement data, such as distances or degrees of movement.

The display module database 275 also adds blood pressure measurements from a blood pressure sensor of display module 100 (e.g., “Blood pressure” column 730). The display module database 275 also adds body temperature measurements (ranging from 98.4 to 98.6) from a thermometer of display module 100 (e.g., “Body temperature” column 735). The display module database 275 also adds respiratory rate measurements (ranging from 11/min to 14/min) from a respiratory rate sensor of display module 100 (e.g., “Respiratory rate” column 740). The display module database 275 also adds global positioning system or “GPS” location coordinates from a GPS module of display module 100 (e.g., “GPS” column 745). Display module database 275 also adds battery percentage measurements pertaining to the percentage of battery power remaining in the display module's battery 255 and/or the wearable band's battery 230 (e.g., “Battery percentage” column 750).

FIG. 8 illustrates an embodiment of sleep graphical user interface (GUI) 280 as executed by display module 100. Sleep GUI 280 may be used by a user of the two-state wearable device 150 to input sleep settings pertaining to wearable band 110.

For example, sleep GUI 280 of FIG. 8 includes a time interface through which a user may select a time at which an alarm should be triggered, or before which a “smart” alarm should be triggered. In an embodiment, sleep GUI 280 indicates that a user has selected that an alarm be triggered at 7:30 AM by selecting “7” using an “Hour” interface 805, selecting “30” using a “Minute” interface 810, and selecting “AM” using an “AM/PM” interface 815. The sleep GUI 280 alternately includes an “input Time” box 820 into which a user may simply type the time in, and into which a user has typed in “7:30 AM.” In some embodiments, inputting time using the “input Time” box 820 may automatically adjust the “Hour” setting 805, the “Minute” setting 810, and the “AM/PM” setting 815 to match the time in the “input Time” box 820. Similarly, in some embodiments, inputting time using the

“Hour” setting 805, the “Minute” setting 810, and the “AM/PM” setting 815 may automatically adjust the “input Time” box 820 to match.

In an embodiment, sleep GUI 280 also allows the user to select a vibration “level” 825 indicating the desired vibration strength. In an embodiment, sleep GUI 280 indicates that a user has selected “high” vibration intensity 840, indicating that perhaps the user is a heavy sleeper. Other vibration level options 825 include a “medium” vibration level 835 and a “low” vibration level 830.

In an embodiment, sleep GUI 280 also includes various options 845. These include a “wait for time” option 850, indicating a length of time during which wearable band 110 should vibrate. Sleep GUI 280 indicates that a user has selected this option 850. Sleep GUI 280 also includes a “vibrate if already awake” option 855, indicating whether or not wearable band 110 should vibrate if it has detected that the user is already awake at the given time (e.g., if wearable band 110 determines that the user is moving through an accelerometer of wearable band 110, or because the user has indicated that he/she is awake through a user interface, or because the user has indicated that he/she is awake by joining the wearable device 150 into the attached state 160). Sleep GUI 280 indicates that a user has not selected this option 855. Sleep GUI 280 also includes an “allow snooze” option 860, indicating whether or not wearable band 110 should apply a “snooze” feature that allows the user to return to sleep for a short period before vibrating again to wake the user up. Sleep GUI 280 indicates that a user has not selected this option 860.

Finally, the sleep GUI 280 also includes a set of “vibrate until” settings 870 indicating a desired time duration of a vibration associated with an alarm. These may be tied to the “wait for time” option 850 that the user has selected in the options 845 section of sleep GUI 280 (and may in some embodiments not appear unless the “wait for time” option 850 has already been selected). The “vibrate until” settings 870 include a “display is connected” setting 875 (not selected in sleep GUI 280) that indicates that wearable band 110 should vibrate until it has been connected to display module 100 (placing the wearable device 150 into the attached state 160 as illustrated in FIG. 1A). The “vibrate until” settings 870 also include a “for 5 minutes” setting 880 (selected in sleep GUI 280) that indicates that wearable band 110 should vibrate for 5 minutes. The “vibrate until” settings 870 also include a “for 1 minute” setting 885 (not selected in sleep GUI 280) that indicates that wearable band 110 should vibrate for 1 minute.

One of ordinary skill will appreciate that sleep GUI 280 is merely an embodiment of a GUI interface that may be employed by wearable device 150. Indeed, according to the purpose of wearable device 150, the settings displayed and adjusted may fit an alternate purpose. For example, a fitness tracker may use different settings that may be adjustable with display module 100.

FIG. 9 is a flow diagram illustrating an embodiment of the operation of a sleep software as executed by display module 100.

In an embodiment, the sleep software 270 first receives an input from the user through the sleep GUI 280 in step 900 (e.g. , sleep GUI 280 of FIG. 8). The sleep software 270 then checks to determine whether display module 100 is connected to wearable band 110 in step 910 (e.g., whether the wearable device 150 is in the attached state 160 or in the detached state 170). If display module 100 is not connected to wearable band 110 (e.g., the wearable device 150 is in the detached state 170), display module 100 may end the sleep software 270 in step 920 or periodically continue to poll regarding whether display module 100 is connected to wearable band 110 in step 910. If it is determined at step 910 that display module 100 is connected to wearable band 110 (e.g., the wearable device 150 is in the attached state 160), the sleep software 270 of display module 100 may transmit the sleep settings determined through the sleep GUI 280 to the wearable band's sleep software 215 in step 930, which may then receive them according to the operations detailed in FIG. 3 in step 940.

While the flow diagram in FIG. 9 shows a particular order of operations performed by certain embodiments, it should be understood that such order is an embodiment (e.g., alternative embodiments can perform the operations in a different order, combine certain operations, overlap certain operations, etc.).

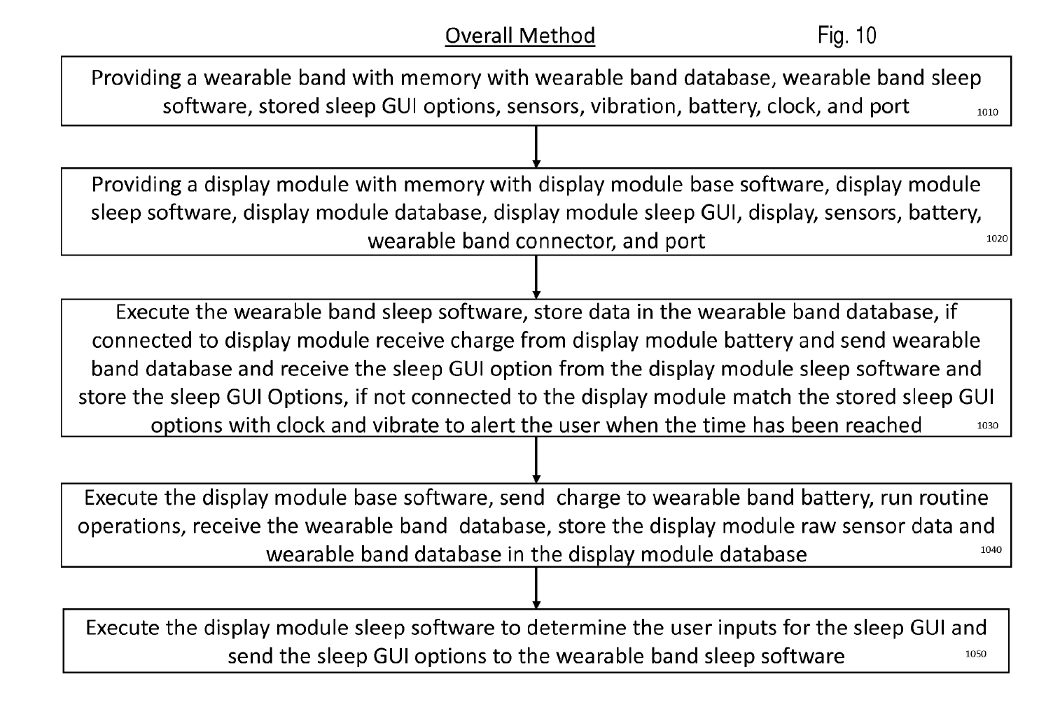

FIG. 10 illustrates an embodiment of an overall method as described herein. In am embodiment, the method of FIG. 10 provides wearable band 110 as described above, which includes a memory 205 with wearable band database 210, wearable band sleep software 215, stored sleep GUI 280 options, sensors 220, vibration 225, battery 230, clock 235, processor 290, and communication/power port/module 130 in step 1010.

The method also provides a display module 100 with memory 260 with display module base software 265, display module sleep software 270, display module database 275, display module sleep GUI 280, display 245, sensors 250, battery 255, wearable band communication/power port/module 240, processor 295, and charging port/module 285 in step 1020.

The method may then execute the wearable band sleep software 215 and store data in the wearable band database 210. If connected to display module 100, wearable band 110 may receive charge from display module battery 255, send the wearable band database 210, receive the sleep GUI 280 sleep settings from the display module's sleep software 270, and store the sleep GUI 280 sleep settings in memory 205. If not connected to display module 100, wearable band 110 may match the stored sleep GUI 280 sleep settings with the wearable band's clock 235 and vibrate (with vibrator 225) to alert the user when a matching time (e.g., an alarm) has been reached in step 1030.

The method may then execute the display module base software 265, send electrical charge to the wearable band's battery 230 from the display module's battery 255, run routine operations, receive at least a subset of the wearable band database 210, and store the display module's raw sensor data and/or wearable band database 210 in the display module database 275 in step 1040.

The method may then execute the display module sleep software 270 to determine the user inputs for the sleep GUI 280 and send the sleep GUI 280 options to the wearable band sleep software 215 in step 1050.

While the flow diagram in FIG. 10 shows a particular order of operations performed by certain embodiments, it should be understood that such order is only an embodiment (e.g., alternative embodiments can perform the operations in a different order, combine certain operations, overlap certain operations, etc.).

Embodiments disclosed herein also relate to an apparatus for performing the operations herein. Such a computer program is stored in a non-transitory computer readable medium. A machine-readable medium includes any mechanism for storing information in a form readable by a machine (e.g., a computer). For example, a machine-readable (e.g., computer-readable) medium includes a machine (e.g., a computer) readable storage medium (e.g., read only memory (“ROM”), random access memory (“RAM”), magnetic disk storage media, optical storage media, flash memory devices).

The processes or methods depicted in the preceding figures can be performed by processing logic that comprises hardware (e.g. circuitry, dedicated logic, etc.), software (e.g., embodied on a non-transitory computer readable medium), or a combination of both. Although the processes or methods are described above in terms of some sequential operations, it should be appreciated that some of the operations described can be performed in a different order. Moreover, some operations can be performed in parallel rather than sequentially.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A wearable device with two states of use, the wearable device comprising:
a wearable band configured to releasably attach a display module such that the wearable band is configurable between an attached state and a detached state, wherein, in the attached state, the wearable band is attached to and in communication with the display module, and in the detached state, the wearable band is separate and apart from the display module, the wearable band comprising:
a wearable band processor configured to execute program instructions and configured to determine when the wearable band is in the detached state or the attached state;
a wearable band battery configured to power the wearable band, at least when in the detached state, and to receive a charge from an external power source;
at least one wearable band sensor configured to perform at least one sensor measurement at least when in the detached state;
a wearable band memory configured to store at least a portion of the wearable band sensor measurements at least when in the detached state; and
a wearable band communication interface configured for transmitting at least a portion of the wearable band sensor measurements to the display module at least when in the attached state.

2. The wearable device of claim 1, wherein the display module, being configured to releasably attach to the wearable band, comprises:
a display module processor configured for executing program instructions and configured to determine whether the display module is attached to the wearable band;
a display module communication interface configured for receiving at least a portion of the wearable band sensor measurements from the wearable band; and
a display module memory configured to receive and to store the wearable band sensor measurements; and
a display module battery configured to power the display module.

3. The wearable device of claim 1, wherein the external power source is the display module battery, and wherein the wearable band battery is configured to receive a charge from the display module battery when in the attached state.

4. The wearable device of claim 1, wherein the external power source is an external connectable battery.

5. The wearable device of claim 1, wherein the display module is a watch face.

6. The wearable device of claim 1, wherein the display module is configured to set at least one setting of wearable band.

7. The wearable device of claim 2, wherein the display may further comprise one or more display module sensors configured for performing one or more display module sensor measurements.

8. The wearable device of claim 1, wherein the wearable band processor is further configured to execute wearable band instructions to generate an alarm output at a predetermined time, the alarm output being one of a vibration of a vibrator of the wearable band or an audio output from a speaker of the wearable band.

9. The wearable device claim 8, wherein the wearable band processor is further configured to execute wearable band instructions to:
process received sleep settings input, the sleep settings input including at least the predetermined alarm time, and
transmit the sleep settings input to the wearable band after determining that the display module is connected to the wearable band.

10. The wearable device of claim 1, wherein the wearable band sensor measurements include sleep measurements related to at least one of sleep quality, sleep duration, sleep movements, sleep patterns, sleep interruptions, sleep pulse, sleep blood pressure, sleep breathing, a time value related to a user falling asleep, or a time value related to a user waking up.

11. A method for utilizing a wearable device with two stages of use, the method comprising:
providing a wearable band configured to releasably attach a display module such that the wearable band is configurable between an attached state and a detached state, wherein, in the attached state, wearable band is attached to and in communication with the display module, and in the detached state, the wearable band is separate and apart from the display module, wherein the wearable band comprises a wearable band processor, a wearable band battery configured to power the wearable band, a wearable band memory, and at least one wearable band sensor;
performing at least one wearable band sensor measurements by the at least one wearable band sensors included in the wearable band;
storing at least a portion of wearable band sensor measurements in the wearable band memory;
determining whether the wearable band is reconnected to the display module; and
transmitting the wearable band sensor measurements from the wearable band memory to the display module.

12. The method of claim 11, further comprising the step of charging a battery of the display module of the wearable device when the display module has been detached from a wearable band of the wearable device, by a power source.

13. The method of claim 11, further comprising the step of setting, using the display module, at least one setting of wearable band.

14. The method of claim 11, further comprising the step of performing at least one display module sensor measurement by at least one display module sensor

15. A wearable device with two states of use, the wearable device comprising:
a wearable band configured to releasably attach a display module such that the wearable band is configurable between an attached state and a detached state, wherein, in the attached state, and in the detached state, the wearable band is separate and apart from the display module, the wearable band is attached to and in communication with the display module, the wearable band comprising:
a wearable band processor configured to execute program instructions and configured to determine when the wearable band is in the detached state or the attached state;
a wearable band battery configured to power the wearable band, at least when in the detached state, and to receive a charge from an external power source;
at least one wearable band sensor configured to perform at least one sensor measurement at least when in the detached state;
a wearable band memory configured to store at least a portion of the wearable band sensor measurements at least when in the detached state;
a wearable band communication interface configured for transmitting at least a portion of the wearable band sensor measurements to the display module at least when in the attached state, wherein the display module, being configured to releasably attach to the wearable band, comprises:
a display module processor configured for executing program instructions and configured to determine whether the display module is attached to the wearable band;
a display module communication interface configured for receiving at least a portion of the wearable band sensor measurements from the wearable band; and
a display module memory configured to receive and to store the wearable band sensor measurements.
